(19) United States
(12) Patent Application Publication
(10) Pub. No.: US 2002/0115117 A1
(43) Pub. Date: Aug. 22, 2002

(54) ERbeta-mediated gene expression

(76) Inventor: Shuk-Mei Ho, Lexington, MA (US)

Correspondence Address: J. Peter Fasse, FISH & RICHARDSON P.C., 225 Franklin Street, Boston, MA, 02110-2804, US

(21) Appl. No.: 09/810,157

(22) Filed: Mar. 15, 2001

Related U.S. Application Data
(60) Provisional application No. 60/189,605, filed on Mar. 15, 2000.

(51) Int. Cl. G01N 33/567; G01N 33/574

(52) U.S. Cl. 435/7.21; 435/7.23

(57) ABSTRACT

An in vitro screening method for identifying a compound that modulates ERβ-mediated cell growth inhibition is disclosed. The method includes: providing a mammalian test cell containing a functional ERβ protein; contacting the test cell with a candidate compound; and detecting an increase or decrease in the expression of an ERβ-regulated gene in the presence of the candidate compound. Compounds that modulate ERβ-mediated cell growth inhibition can promote or inhibit this process. In some embodiments, the test cell contains no detectable ERα protein. The ERβ-regulated gene can be, e.g., the genes encoding receptor-like tyrosine kinase (RYK), 5-hydroxytryptamine A1 receptor (E2c), BCL-2 related A1, embryonic growth/differentiation factor, IL-12, TL1309, or IFN-α/β receptor.

This patent application claims priority from provisional application serial No. 60/189,605 filed on Mar. 15, 2000, which is incorporated herein by reference in its entirety.

### FEDERALLY SPONSORED RESEARCH

Work on the invention was supported in part by U.S. Army grant no. DAMD17-98-1-8606. Therefore, the government has certain rights in the invention.

### TECHNICAL FIELD

This invention relates to molecular biology, urology, neurobiology, endocrinology, and oncology.

### BACKGROUND

Estrogen, a steroid hormone, is involved in growth, differentiation and function of various target tissues in the female and male reproductive systems, including the male prostate gland. Estrogen is also plays certain roles in the central nervous system and the cardiovascular system. Estrogen crosses cell membranes and exerts its effects through binding with high affinity to an estrogen receptor (ER) in the cell nucleus.

Historically, the actions of estrogens/antiestrogens were thought to be mediated by the classical ER, now called ERα. In human and rodent prostates, ERα is localized in the stromal compartment and basal epithelial cells of the prostate (Prins et al., Endocrinology, 138:1801-1809, 1997; Bonkhoff et al., Am. J. Pathol., 155:641-647, 1999; Wernert et al., Virchows Arch. A. Pathol. Anat. Histopathol, 412:387-391, 1988; Ehara et al., Prostate, 27:304-313, 1995; Kirschenbaum et al., J. Androl, 15:528-533, 1994; Hiramatsu et al., Histopathology, 28:163-168, 1996). Because ERα is not expressed in the normal glandular epithelium of rat or human prostate (Hartley-Asp et al., Mutat. Res., 143:231-235, 1985; Lau et al., Endocrinology, 139:424-427, 1997; Bonkhoff et al., Am. J. Pathol. 155:641-647, 1999; Ehara et al., Prostate, 27:304-313, 1995; Kirschenbaum et al., J. Androl., 15:528-533, 1994, Hiramatsu et al., Histopathology, 28:163-168, 1996; Rohlff et al., Prostate, 37:51-59, 1998), it has seemed likely that the action of estrogen/antiestrogen on normal prostate epithelial cells (PrECs) is indirect, likely mediated via estrogen-induced stromal factors.

In 1996, the cloning of ERβ, a second subtype of ER, was reported. ERβ was found to be expressed at high levels in the epithelial compartments of the rat prostate (Mosselman et al., FEBS Lett. 392:49-53, 1996; Kuiper et al., Proc. Natl. Acad. Sci. USA 93 :5925-5930, 1997). Although ERβ shares high homology with the classical ER (ERα), it has been suggested that the two ER subtypes regulate different sets of cellular functions (Paech et al. Science, 277:1508-1510, 1997; Montano et al., J. Biol. Chem., 273 :25443-25449, 1998.).

Several investigators have reported ERα expression in human prostate cancer cell lines, including LNCaP, PC-3 and DU-145 (Carruba et al., Cancer Res., 54:1190-93. 1994; Castagnetta et al., Endocrinology, 136:2309-2319, 1995), but others have reported failure to find such results (Hobisch et al., J. Pathol, 182:356-361, 1998.). Observations on ERα expression in prostate cancer specimens also have conflicted. Bonkhoff and co-workers reported ERα expression to be infrequent in low-to-moderate grade adenocarcinoma, but common in high grade and metastatic cancers (Bonkhoff et al., supra).

Similarly, published information concerning the distribution of ERβ in normal and malignant human PrECs has conflicted. At least one study reported lack of ERβ expression in human prostate tissues (Bonkhoff et al., supra), but other published reports noted expression of this receptor subtype in basal epithelial cells of the human (Lau et al., Proc. Amer. Assoc. Cancer Res. Annual Meeting, 40:637-638, 1999; Sinisi et al., Proc. Endocrine Soc., 81^{th} Ann. Mtg, P1-611, 1999; Taylor et al., Proc. Endocrine Soc. 81^{th} Ann. Mtg, P1-228, 1999).

In view of such conflicting reports, the roles played by estrogens in the neoplastic transformation of PrEC, and in prostatic carcinoma progression and treatment, have been controversial. Exposure of human or rodent to estrogens induces a proliferative lesion, squamous metaplasia, and in their prostates (Sugimura et al., Hum. Pathol, 19: 133-139, 1988; Yonemura et al., Acta. Anat. (Basel), 153:1-11, 1995; Triche et al., Lab. Invest, 25:596-606, 1971; Levine et al., J. Urol., 146:790-793, 1991), while prolonged treatment of Noble rats with androgen plus estrogen causes a high incidence of prostate adenocarcinoma in the dorsolateral prostates of the treated animals (Noble et al., Cancer Res., 40:3547-3550, 1980; Drago, Anticancer Res., 4:255-256, 1984; Leav et al., Prostate, 15:23-40, 1995; Bosland et al., Carcinogenesis, 16: 1311-1317, 1995). Paradoxically, DES, TAM and other estrogens have been used as treatment regimens for advanced metastatic prostate cancer (Ahmed et al., Int. Urol. Nephrol, 30(2): 159-64, 1998; Bergan et al., Proc. Amer. Soc. Clin Oncol, 14:A637, 1995; Bergan et al., Clinical Cancer Res., 5:2366-2373, 1999; Klotz et al., J. Urol., 161:169-172, 1999; Smith et al., Urology, 52:257-260 1998). In addition to acting as chemical castration agents, both estrogen and antiestrogen are believed to exert direct growth inhibitory effects on prostatic cancer cells via induction of apoptosis or cell cycle arrest (Brehmer et al., J. Urology, 108:890-896, 1972; Hartley-Asp et al., Mutat. Res., 143:231-235, 1985; Schulze et al., Prostate, 16:331-343, 1990; Robertson et al., J. Natl. Cancer Inst., 88:908-917, 1996; Landstrom et al., Int. J. Cancer, 67:573-579, 1996). Details of how estrogens and antiestrogens elicit these responses has remained unclear.

### SUMMARY

The invention is based on the discovery that: (1) normal human prostate epithelial cells (PreCs) and certain prostate cancer cells express exclusively ERβ, (2) estrogen/anti-estrogen action in normal and malignant prostate epithelial cells is mediated by ERβ, and (3) anti-estrogen-induced cell growth inhibition (cell death/apoptosis/cell cycle arrest) in prostate cancer cells is mediated through an ERβ signaling mechanism. In addition, there are seven genes in prostatic cancer cells that display ERβ-mediated up-regulation in response to the antiestrogen ICI 182,780. Furthermore, ER-β is exclusively expressed in the nuclei of basal cells in normal prostate and down regulation of ERβ expression occurs during prostatic carcinogenesis. Specifically, ER-β is not expressed in high-grade dysplasia or in grade 4/5 neoplasms, but is expressed in grade 3 lesions. ER-β is also expressed in metastatic lesions.

Based, in part, on these discoveries, the invention features in vitro screening methods for identifying a compound that modulates ERβ-mediated cell growth inhibition. Some of these methods include: (a) providing a mammalian test cell containing a functional LRβ protein; (b) contacting the test cell with a candidate compound; and (c) detecting an increase or decrease in the expression of an ERβ-regulated gene in the presence of the candidate compound, compared to the level of expression of the gene in the absence of the candidate compound. Compounds that modulate ERβ-mediated cell growth inhibition can promote or inhibit this process. In some embodiments, the test cell contains no detectable ERα protein. In some embodiments, the test cell is derived from a prostate cell, a neuronal cell, an ovarian cell, a breast cell, a cardiovascular cell or a bone progenitor cell. In some embodiments, the test cell is engineered to contain an exogenous ERβ gene, i.e., an ERβ transgene. The ERβ-regulated gene can be, e.g., receptor-like tyrosine kinase (RYK), 5-hydroxytryptamine A1 receptor (E2c), BCL-2 related A1, embryonic growth/differentiation factor, IL-12, TL1309, or IFN-α/β receptor. In some embodiments, a reporter gene coding sequence is operably linked to an ERβ-regulated promoter or enhancer. A multiplicity of ERβ-regulated genes can be monitored to detect an increase or decrease in expression, for example by using an oligonucleotide array or subtractive hybridization technology.

As used herein, “cell growth inhibition” means cell death, apoptosis, or cell cycle arrest.

As used herein, “detecting an increase” in expression of a gene means detecting an increase from any baseline level, including zero expression.

As used herein, “detecting a decrease” in expression of a gene means detecting a decrease to any reduced level, including zero expression.

As used herein, “ERβ-regulated gene” means a naturally occurring ERβ-regulated gene or a gene construct containing any coding sequence operably linked to one or more ERβ-regulated gene expression control elements.

Unless otherwise defined, all technical and scientific terms used herein have the same meaning as commonly understood by one of ordinary skill in the art to which the invention pertains. In case of conflict, the present application, including definitions will control. All publications, patent applications, patents, and other references cited herein are incorporated by reference in their entirety.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a table summarizing the results of RT-PCR analyses of ERα, ERβ, progesterone receptor (PR), pS2, and androgen receptor (AR) mRNAs among normal and malignant prostatic epithelial cells. Total RNAs were extracted and reverse-transcribed. The resultant cDNAs were subjected to PCR analyses under optimized conditions. The amplified products were run into 2% agarose gel with eithidium bromide. Three individual experiments were performed. A summary of the data from RT-PCR analyses performed in three individual experiments (+, detectable level of expression; −, undetectahle level of expression).

FIGS. 2A-2D are graphs summarizing data on effects of antiestrogens and estrogens on cell growth of DU145 cells. Cells (5×10³ cells per well) were plated in triplicate wells onto a 24-well plate. After 24 hours for cell attachment, cells were treated for 4 days with antiestrogens [(A) ICI and (B) 4OH-TAM] or estrogens [(C) 17β-estradiol and (D) DES] at various concentrations as indicated. Cells treated with vehicle (absolute ethanol) were used as control. The number of viable cells at the end of 4 days of treatment was determined by the trypan blue exclusion method. Three individual experiments were performed. (Columns, means; bars, S.D.; n=9; *, p<0.001 compared to control (shaded column).

FIGS. 3A-3D are graphs summarizing data on effects of antiestrogens and estrogens on cell growth of PC-3 cells. Cell growth assay was described in FIG. 3. (A) ICI. (B) 4OH-TAM. (C) 17β-estradiol. (D) DES. Columns, means; bars, S.D.; n=9; * ,p<0.001; #, p<0.01; +, p<0.05; compared to control (shaded column).

FIG. 4 is a graph summarizing data on reduction of ICI-induced inhibition of DU145 cell growth by ERβ antisense oligonucleotide (ODN). Cells were treated with ICI at 10⁻⁶ M for 4 days in the presence of 2.5 μM antisense, sense or mismatch ODNs. Columns, means; bars, S.D.; n=16; *,p<0.001 compared to control without ODN treatment.

FIG. 5 is a graphic representation of the results of a competitive ELISA assay and illustrates the concentration-dependent competition of the immunizing peptide (ERβC) with a GC-17 antibody. Preincubation with 4 mg/ml of the control N-terminus peptide (ERβN) and GC-17 failed to compete out the binding to the recombinant ER-β peptide, demonstrating the specificity of the antibody. The GC-17 antibodies, bound to recombinant ER-β protein on ELISA plates, were recognized by alkaline phosphatase conjugated anti-rabbit IgG antibodies. The whole complexes were visualized by incubation with p-nitrophenyl phosphate. The optical density at 405 nm was measured. The entire assay was done 4 times. The columns represent the standard deviation. w/o=GC-17 preincubated without peptide antigen.

FIGS. 6A and 6B are representations of the structures of various phenyl vinyl substituted estrogens (6A) and a Table (6B) showing the effects of various phenyl vinyl substituted estrogens (a type of candidate compound) on cell proliferation in DU-145 cells. %Cell# indicates the percentage of cells surviving after treatment with a candidate compound compared to untreated controls. MW is the molecular weight of the candidate compound.

FIGS. 7A and 7B are representations of the amino acid and nucleic acid sequences of human estrogen receptor beta.

### DETAILED DESCRIPTION

Any cultured, ERβ-containing mammalian cells can be employed as mammalian test cells in new in vitro screening methods for identifying compounds that modulate ERβ-mediated cell growth inhibition. The cells can be in primary cell culture or in an established line such as a PrEC prostate epithelial cell line. An example of an established PrEC cell line useful in the invention is BPH-1 (described in Hayward et al., In Vitro Cell Dev. Biol. Anim., 31:14-24, 1995; DSMZ- Deutsche Sammlung von Mikroorganismen und Zelllkulturen GmbH, cat. # ACC143), a nontumorigenic, SV40-immortalized, highly differentiated human prostate epithelial cell line. Another example of a useful, established prostate epithelial cell line is available commercially as PrEC™ (Clonetics). An alternative to using an established cell line is to isolate and maintain highly enriched populations of normal PrECs in primary cultures, for example, as described below, in the “Examples.” Also useful as test cells are prostatic cancer cell lines such as LNCap (ATCC no.CRL-1740), PC-3 (ATCC no. CRL-1433), or DU-145 (ATCC no. HTB-81). A useful non-prostate cell line is ovarian cell line SKOV-3.

Since estrogens are involved in the development and maintenance of the neuronal tissues, identification of compounds that reduce (rather than promote) ERβ-mediated apoptosis in neuronal tissues may be desirable, and can be identified using an in vitro screening method of the invention. In some embodiments of the invention designed to identify compounds particularly suitable for neurological use, a test cell of neurological origin is used. Test cells having other origins, e.g., cardiovascular cells or bone progenitor cells, can employed as appropriate, where identification of compounds to treat particular tissues is sought.

The necessary ERβ protein in the cell can be expressed from an endogenous gene or an exogenous (engineered) gene. The ERβ gene can be carried on a transient expression vector. Preferably, however, an exogenous ERβ gene is stably integrated, so that repeated transfections are not a necessary part of the assay protocol. In some embodiments of the invention, an engineered ERβ gene is placed under the control of an inducible promoter. Testing in the presence and absence of the inducer provides a convenient way to confirm that an observed effect of a candidate compound is ERβ-mediated. If the effect is ERβ-mediated, it will be reduced or absent when the test cell is contacted with the candidate compound in the absence of the inducer.

The test cell can contain other natural ER subtypes, e.g., ERα, or genetically engineered ER molecules in addition to ERβ. In addition, the test cell can contain an estrogen receptor co-regulator (co-activator or co-repressor). However, a test cell containing exclusively ERβ (and optionally an estrogen receptor co-regulator) advantageously reduces the probability of “false positives,” i.e., candidate compound-dependent gene expression changes that are not ERβ-mediated. To promote reliability and consistency in screening assay results, levels of one or more ER subtypes in test cell lines can be assayed periodically. Such assays can be based on ER protein level, mRNA level, or both. ER protein assays and ER mRNA assays can be performed by a person of ordinary skill in the art, using conventional methods and materials.

The ERβ-regulated gene whose expression is monitored in an in vitro screen method of the invention can be any ERβ-regulated gene in the test cell, e.g., receptor-like tyrosine kinase (RYK), 5-hydroxytryptamine A1 receptor (E2c), BCL-2 related A1, embryonic growth/differentiation factor, IL-12, TL1309 or IFN-αβ receptor. Expression of the gene can be monitored at any level, e.g., mRNA or protein.

In some embodiments of the invention, a reporter gene coding sequence is engineered into the ERβ-regulated gene or driven by an ERβ-regulated promoter-enhancer such as AP-1, ERE or electrophilic response element. For example, a fusion protein containing all or part of the ERβ-regulated gene product and all or part of a reporter gene product could be expressed in the assay. The reporter gene coding sequence is chosen to provide an assay “tread-out” that is more convenient or more rapid (or both), relative to an assay read-out based on the native ERβ-regulated gene. Examples of useful reporter genes include those that encode a fluorescent protein, e.g., green fluorescent protein (GFP) or a variant thereof; a bioluminescent protein, e.g., luciferase; a calorimetric enzyme, e.g., β-galactosidase. Various reporter genes are known in the art and can be used in gene constructs for assaying expression of the ERβ-regulated gene. See, e.g., Cubitt et al., 1995, “Understanding, improving and using green fluorescent proteins,” Trends in Biochemical Science 20:488-455. The GFP domain of a fusion protein can be encoded by a mammalian-optimized version of a GFP cDNA. See, e.g., Cormack et al., 1997, Microbiology 143:303-311.

An in vitro screening method of the invention can involve detecting an increase or decrease in the expression of a multiplicity of ERβ-regulated genes, rather than a single ERβ-regulated gene. This can be accomplished, for example, by contacting the test cell's RNA with an oligonucleotide array (see, e.g., Behling et al., 2001, Proc. Natl. Acad. Sci. USA, 98:1176-1181; and Hawthorne et al., 2000, Circulation, 102:3046-3052, 2000) or b employing subtractive hybridization technology.

In addition to being useful for screening synthetic compounds for potential use as pharmaceutical agents, the methods of the invention can be used to test dietary components, e.g., red wine, for the presence of estrogen mimics that trigger ERβ-mediated processes, thereby affecting physiological function positively or negatively. Similarly, the methods of the invention can be used to test environmental pollutants for the presence of estrogen mimics that may pose health risks involving ERβ-mediated processes.

Various compounds can be screened using the new methods. The ligand binding cavity of ERβ is different from that of ERα and therefore it will be possible to develop receptor-specific ligands that may form the basis of novel pharmaceuticals with better in vivo efficacy and side effect profile than currently available drugs to target disease cells that express ER-β only. Several broad classes of compounds can be screened. Useful compounds include those that specifically bind to ERβ or are predicted to bind to ERβ. Compounds that specifically bind to ERβ and do not have significant binding to ERα are also useful. Such compounds are identified using methods known in the art. Other useful compounds are those known to have estrogen or anti-estrogen activities in one or more tissues or cell types (e.g., prostate or nervous system).

Other compounds that can be screened include ER-β agonists such as anti-estrogen therapeutics (tamoxifen, ICI 164,384, ICI 182,780 (see, e.g., Van Den Bemd et al., 1999, Biochem. Biophys. Res. Commun., 261(1):1-5; PMID: 10405313), and diethylstilbestrol), xenocompounds such as environmental pollutants and industrial waste, dental compounds (2′,3′,4′,5′-tetrachlorobiphenyl-ol, bisphenol A, 4-tert-octylphenol, phytoestrogens (compounds present in the plants, mostly as part of our diet), Quercetin, genistein, resveratrol, and natural estrogens and metabolites (e.g., estradiol, and 17 epiestriol). ER-β antagonists can also be used. Examples include: 5,11-cis-diethyl-5,6,11,12-tetrahydrochrysene-2,8-diol (see, Meyers et al., 1999, J. Med. Chem., 42(13):2456-68 (PMID: 10395487) and Sun et al., 1999, Endocrinology, 140(2):800-4 (PMID: 9927308).

Based on the test results described herein, prostate carcinogenesis is characterized by a loss of ERβ expression at the protein and transcript levels in high grade dysplasia, a reappearance of ERβ expression in grade 3 cancers, and the diminution/absence of the receptor in grade 4/5 neoplasms. ERβ is also expressed in most bone and lymph metastasis of prostate cancer. Therefore, in some embodiments of the invention a prostate dysplasia or cancer cell is assayed for ERβ. This assay is useful for diagnosis (e.g., staging) of prostate cancer, determining the prognosis for an individual who has prostate cancer, and determining an appropriate treatment for an individual who has prostate cancer.

One method of assaying ERβ is using an antibody that specifically binds the receptor. Methods of generating such an antibody are described herein.

### EXAMPLES

The following examples are meant to illustrate, but not limit the invention.

### Example 1

### Establishment of Normal Prostate Epithelial Cells in Primary Cultures

Tissue specimens used for generating primary cultures of normal Prostate Epithelial Cells in Primary Cultures (PrECs) were obtained from patients undergoing transrectal ultrasound-guided biopsies of the prostate for standard clinical indications. All patients contributing biopsy material were invited to participate in a prospective tissue acquisition study approved by the local IRB. Written informed consent was obtained prior to biopsy from participating patients. From each participant, one biopsy core was obtained from the peripheral zone of the prostate, placed on a sponge pad soaked in sterile saline, a 1-2 mm section was excised from the mid-portion of the core, and suspended in 5 ml of culture medium (described below). The two remaining ends of the core were inked at the ends opposite the sectioned mid-portion piece, placed in 10% formalin, and processed for histology. Histological examination of the end pieces of a biopsy core allowed us to determine the histologic nature and the homogeneity of the core. Only specimens judged to be histologically normal, with no hyperplastic or neoplastic tissue contamination, were used to establish primary cultures of normal PrECs.

Each harvested tissue specimen was then washed three times with Hank's balanced salt solution (HBSS) and cut into 5-7 smaller pieces. The pieces were suspended in 2 ml freshly prepared growth medium (see below) and transferred to a 60-mm Falcon culture dish (Becton Dickinson, Lincoln Park, N.J.) coated with Type I rat tail collagen (Collaborative Biomedical Products, Bedford, Mass.). An epithelial cell selection medium (the growth medium) was used to obtain enriched populations of PrECs. The epithelial cell selection medium was essentially as described in Bright et al., Cancer Res., 57:995-1002, 1997, consisting of: keratinocyte serum-free medium with 25 μg/ml bovine pituitary extract, 5 ng/ml epidermal growth factor, 2 mM L-glutamine, 10 mM HEPES buffer, P/S (100 U/ml Penicillin and 100 μg/ml Streptomycin), 5.5 μl/ml fungizone, 20 ng/ml cholera toxin and 1% heat-inactivated fetal bovine serum (FBS). All culture reagents were obtained from Life Technologies (Grand Island, N.Y.) except for FBS, which was purchased from Sigma (St. Louis, Mo.). The culture was incubated at 37° C. in a 5% CO₂ atmosphere without disturbance for 7 days to allow epithelial cells to grow out of the tissue pieces. Culture medium was then routinely replaced every 4 days until cell culture reached approximately 80% confluence. The cells were split once before they were used for RNA extraction.

In addition to primary cultures obtained from biopsy explants, a batch of normal human prostate epithelial cells (PrEC™) were purchased from Clonetics Co. (San Diego, Calif.). The PrEC™ cells were cultured in the PrEGM medium supplemented with SingleQuots™ (Clonetics Co., San Diego, Calif.) according to the manufacturer's recommended protocol.

### Example 2

### Maintenance of Established Prostatic Cell Lines

All culture reagents were obtained from Life Technologies (Grand Island, N.Y.) and FCS from Sigma (St. Louis, Mo.) except otherwise specified. BPH-1 (described in Hayward et al., In Vitro Cell Dev. Biol. Anim., 31:14-24, 1995), a nontumorigenic, SV40-immortalized, highly differentiated human prostate epithelial cell line was provided by Dr. Simon Hayward, Univ. of California at San Francisco. This cell line was maintained in RPMI1640 with 10 mM HEPES, 1 mM sodium pyruvate, 2 mM L-glutamine, 4.5 g glucose/L and 1.5 g sodium bicarbonate/L (ATCC, Rockville, Md.) plus 5% heat-inactivated FBS, ITS+™ (Insulin-Transferrin-Selenium mixture, Collaborative Biomedical Research, Bedford, Mass.), and P/S. Three human prostate cancer cell lines (DU145, PC-3 and LNCaP) were purchased from ATCC. For routine maintenance, DU145 and PC-3 cells were grown in DMEM/F-12 supplemented with heat inactivated FBS, 2 mM L-glutamine, 1 mM sodium pyruvate, 0.1 M non-essential amino acids, P/S, 0.05 mM β-mercaptoethanol (Sigma Co), and 1% ITS+™. LNCaP were maintained in the same medium used for BPH-1 except that ITS+™ was left out from the medium. All cell cultures were incubated at 37° C. under a 5% CO₂ atmosphere.

### Example 3

### RNA Isolation and Reverse Transcriptase-PCR (RT-PCR)

Total cellular RNA was isolated using RNA Stat-60 reagent (Tel-Test Inc., Friendwood, Tex.) according to protocols provided by the manufacturer. The quality of each total RNA sample was checked and controlled by the following steps: 1) measurement of optical density, 2) running of a denaturing RNA gel capable of detecting possible RNA degradation, as judged by the integrity and intensity of the 18S and the 28S ribosomal RNA signals, and 3) conducting a semi-quantitative RT-PCR for the 18S ribosomal RNA at low cycle numbers. One μg of total cellular RNA was reverse-transcribed using the GeneAmp RNA PCR kit (Perkin-Elmer, CT) and 2 μl of the resulting cDNA was used in each PCR.

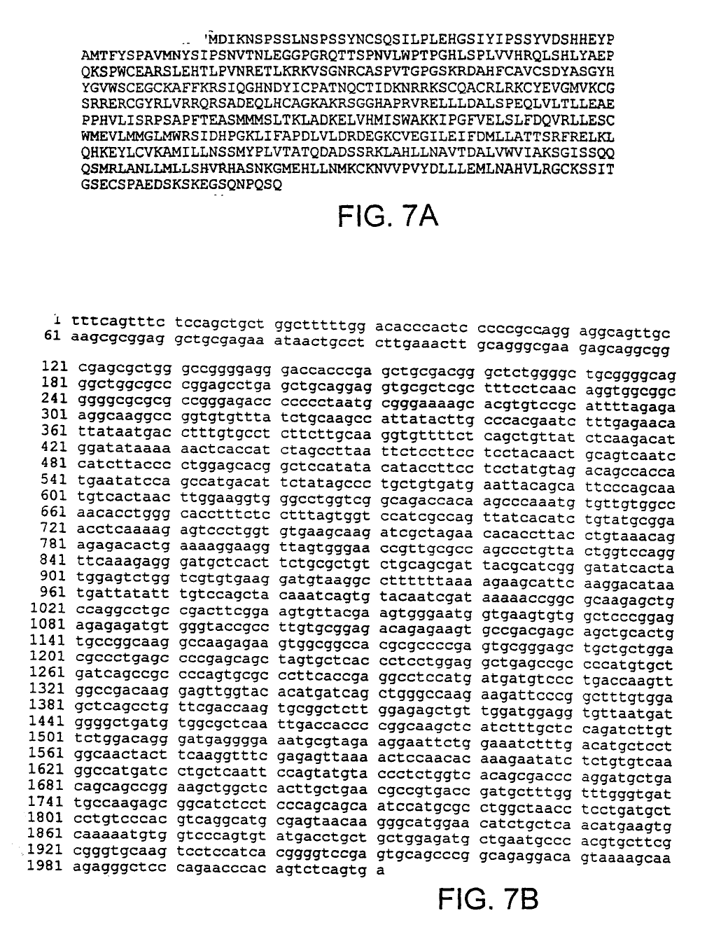

Intron-spanning primers were either obtained from published literature or designed using the Primer3 Output program (http://www.genome.wi.mit.edu/cgi-bin/primer/primer3.cgi). Primer sequences for GAPDH (GenBank Acc. No. M33 197), ERα (GenBank Acc. No. M12674), ERβ (GenBank Acc. No. AF051427, see FIG. 7A which illustrates an amino acid sequence of ERβ, and FIG. 7B, which illustrates an mRNA nucleic acid sequence for ERβ), PR (GenBank Acc. No. M1576), and AR (GenBank Acc. No. L29496) are given in Table 1 (below). All PCR conditions were optimized for quantification of relative message contents under non-saturating conditions. Preliminary experiments were conducted to ensure linearity for all semiquantitative procedures. Hot start PCR using AmpliTaq Gold DNA polymerase (Perkin-Elmer, CT) was employed in all amplification reactions. The enzyme was activated by pre-heating the reaction mixtures at 95° C. for six minutes prior to PCR. This protocol was chosen to minimize non-specific product amplification. The routine PCR program was 30 cycles of 1 minutes at 94° C., 1 minutes at 60° C. (annealing temperature) and 1 minute at 72° C. with the following modifications: (1) amplification for ERβ cDNA used an annealing temperature of 58° C., (2) amplifications of ERα cDNA and AR cDNA were carried out at an annealing temperature of 55° C., (3) cycle-number for ERα cDNA amplification was set at 35, and (4) GAPDH cDNA was amplified at 26 cycles. GAPDH cDNA levels served as a loading control. Amplification of the correct sequence was verified by direct DNA sequencing of each PCR product from at least two different samples.

### Example 4

### Cell Treatments

Treatment of DU145, PC-3 and LNCaP cells with demethylating agents The three prostatic cancer cell lines were seeded at a density of 1 cells per ml medium in 25 cm² culture flasks, allowed to attach during a 24 hour period, and exposed to two demethylating agents separately. The demethylating agents were added daily in aqueous solution. 5′azacytidine was added at final concentrations of 2.5 μM and 5 μM and 5′aza-2′-deoxycytidine at 0.5 μM and 0.75 μM, respectively. Culture medium was changed every four days and cells were subjected to a total of 8 days of demethylating agent treatment. At the end of the treatment period, the medium was removed, and cellular RNA extracted for RT-PCR.

Treatment of DU145 and PC-3 cells with estrogens/antiestrogens

Cells were seeded at a density of 5×10³ per ml into 24-well plates (Falcon, Becton Dickinson Labware, Lincoln Park, N.J.) in a final volume of 1 ml culture medium with 5% charcoal-stripped FBS. Twenty-four hours following seeding, triplicate wells of cells were treated in with 1 μM, 10 μM and 100 μM of estradiol-17β (E₂), diethylstilbestrol (DES), 4′-hydroxytamoxifen (4OH-TAM) or ICI 182,780 (ICI). E₂, DES, and 4OH-TAM were purchased from Sigma Co. (St. Louis, Mo.) and ICI was a gift from Zeneca Pharmaceuticals (Macclesfield, United Kingdom). Estrogens and antiestrogens were dissolved in absolute ethanol (Sigma Co., St. Louis, Mo.) and added to the media daily. Cell cultures not treated with estrogenic compounds received absolute ethanol as a vehicle control. Total additive ethanol concentrations never exceeded 0.2% throughout the culture period. The cells were re-fed with freshly prepared medium every other day. At the end of a 4-day treatment period, cells in each well were trypsinized and cell count determined by direct counting using the Trypan blue exclusion method. All treatment experiments were repeated at least three times to generate statistically relevant data.

Treatment of DU145 Cells with ICI and ERβ Antisense Oligonucleotide (ODN)

DU145 cells (5×10³ cells per well) were plated in 24 well-plates (Falcon, Becton Dickinson Labware, Lincoln Park, N.J.). After allowing 24 hours for cell attachment, cell cultures were treated in triplicate with 1 μM ICI in the presence of 2.5 μM ERβ antisense, sense or mismatch ODNs for 4 days. The ERβ antisense ODN, an 18-mer, was designed to recognize the first translation start site on the ERβ mRNA and its immediate 5′flanking region (Table 2, below). The nucleotide sequence of sense ODN is complementary to those of ERβ antisense ODN (Table 2). Based on the sequence of ERβ antisense ODN, 5 nucleotides were scrambled to generate a mismatch ODN that retains the same GC ratio of the ERβ antisense ODN (Table 2). Both the sense and the mismatch ODNs served as controls for the antisense ODN. In all three ODNs, the first and the last 3 nucleotides were phosphorothioate-modified to increase their stability in cellulo. Number of viable cells in each well was determined by direct counting using the Trypan blue exclusion method after a 4-day treatment period. At least three individual experiments were performed to obtained statistically relevant data.

Statistical analysis was performed by using Student SYSTAT software (Course Technology, Inc., Cambridge, Mass.). Data was analyzed by one-way ANOVA followed by the Tukey post-hoc test and a 95% confidence limit was used for all comparisons among treatment groups.

### Example 5

### AR, ERβ, PR and pS2 mRNA Expression

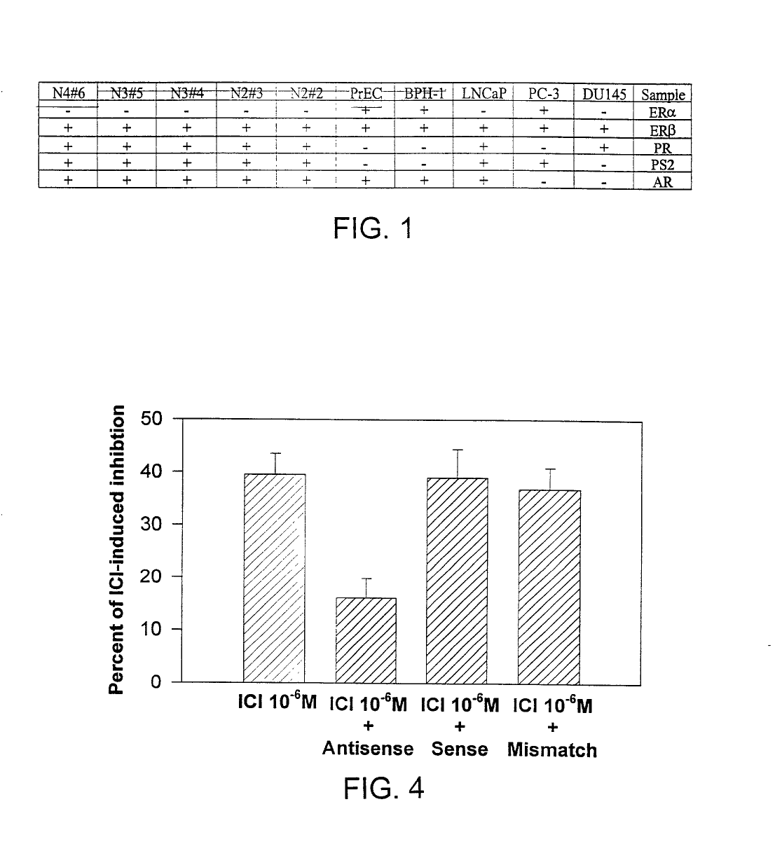

Five primary cultures of normal PrECs (N4#6, N3#5, N3#4, N2#3, N2#2) were established from ultrasound-guided peripheral zone biopsies over a period of 18 months. Upon histological examination, the biopsy cores were all judged to contain only normal prostatic tissue with no BPH or cancerous foci contamination. The primary cell cultures were all early passages (second or third), cobblestone in appearance with no visible fibroblast contamination. Semiquantitative RT-PCR analyses (FIG. 1) demonstrated that normal PrEC cultures retained high levels of androgen receptor (AR) mRNA expression, which usually disappeared in late passage normal PrEC primary cultures or in established PrEC. All five cultures of normal PrECs expressed uniform levels of ERβ RNA, and transcripts of the estrogen responsive genes, PR and pS2. In contrast, expression of ERα mRNA was noticeably absent in all five cultures even when high cycle number PCR (>42 cycles) was used to amplify the cDNA. PrEC™ (Clonetics Co), a commercially prepared normal PrEC culture, and BPH-1, a SV-40 immortalized prostatic epithelial cell line, expressed both ERα and ERβ, but no PR or pS2 transcripts in PrEC™ and only minimal level of PR mRNA in BPH-1.

All three prostatic cancer cell lines, DU145, PC-3 and LNCaP, express ERβ mRNA (FIG. 1). In contrast, ERα mRNA was expressed only in the PC-3 cells. Interestingly, PR transcripts were detected only in DU145 and LNCaP cells, and not in PC-3 cells. Messages of pS2 were found in PC-3 and LNCaP cells, but not in DU145 cells. In accordance with reports in the literature, AR mRNA expression was only noted in LNCaP cells.

When RT-PCR analyses were conducted for ERα mRNA semiquantification in PrEC™ (Clonetics Co), BPH-1 or PC-3 cells we noticed that, in addition to the expected PCR product, a smaller PCR product was co-amplified. Sequencing analysis revealed that this smaller PCR product was derived from an ERα mRNA variant which had whole exon 2 deleted.

Prior to exposure to demethylating agents, ERα and pS2 transcripts were not detected in RNA samples prepared from DU145 cells (FIG. 1). After the 8-day treatment with 5′-aza-cytidine (2.5 μM and 5 μM) or 5′-aza-2′deoxycytidine (0.5 μM and 0.75 μM), DU145 cells regained expression of both transcripts. Interestingly, the absence of AR mRNA expression in DU145 cells was not reversed by treatment with demethylating agents. Exposure of LNCaP cells to demethylating agents also reactivated ERα mRNA expression.

### Example 6

### Effect of Anti-estrogens and Estrogens on Cell Growth of DU145 and PC-3

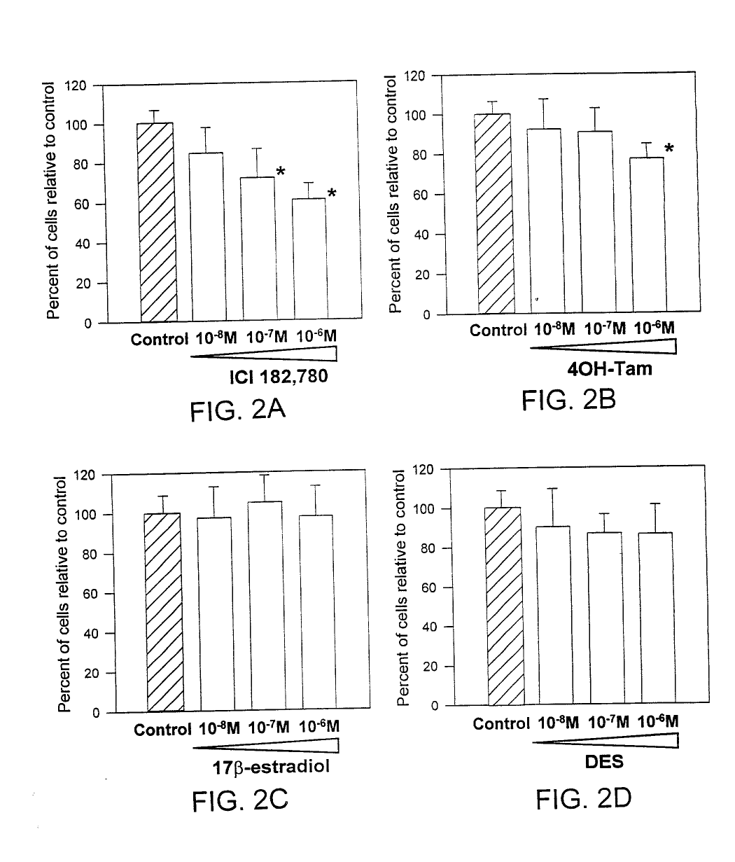

Cell growth analyses showed that the growth of DU145 cells, which only expressed ERβ mRNA, was adversely affected by the antiestrogens, ICI and 4OH-TAM (FIGS. 2A and 2B). A dose-dependent inhibition of cell numbers was observed in cultures exposed to ICI for 4 days when compared to control cultures treated with vehicle (absolute ethanol). A 40% reduction (p<0.001) in the cell numbers was achieved with an ICI dose of 1 μM. A similar growth inhibitory response was observed when DU145 cells were treated with 4OH-TAM. However, cell number reduction achieved with 1 μM of 4OH-TAM was only around 25% (p<0.001). In contrast, exposure of DU145 cells to estrogens (E2 and DES) did not affect cell growth in 4-day exposure experiments (FIGS. 2C and 2D).

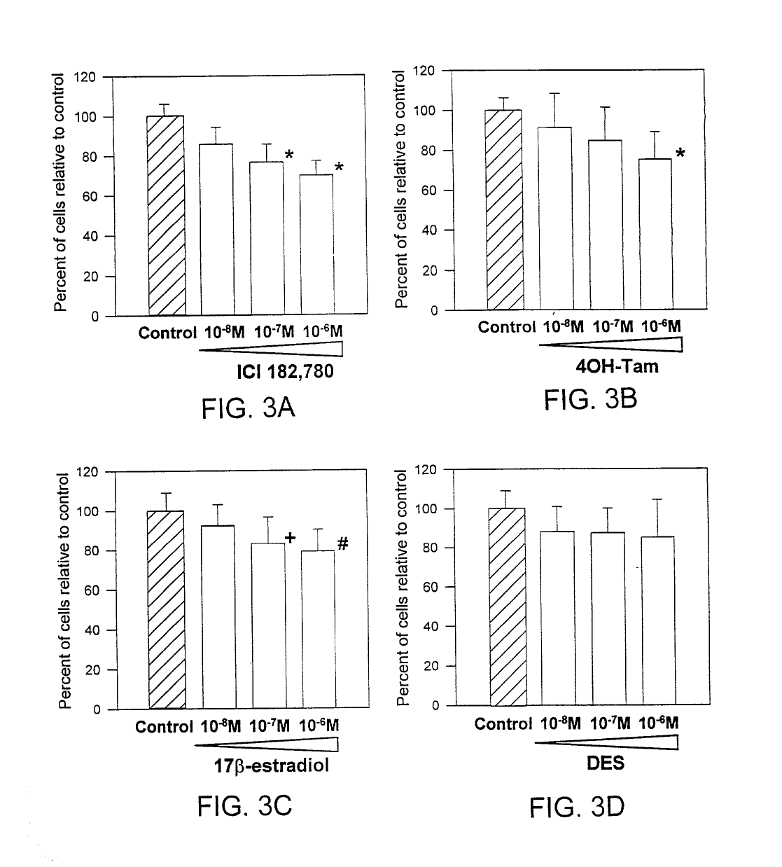

When PC-3 cells, which expressed transcripts of both ER subtypes, were exposed to antiestrogens (ICI and 4OH-TAM) a 25-30% reduction in cell growth was noted in cultures treated with 1 μM or 10 μM of ICI, or with 1 μM of 4OH-TAM (p<0.001, FIGS. 3A and 3B). Furthermore, exposure of PC-3 cells to E2 at 1 μM or 10 μM concentrations also induced inhibition of cell growth (p<0.05 and p<0.01, respectively; FIG. 3C). Treatment with DES at the various concentrations did not elicit statistically significant cell growth inhibition in PC-3 cells (FIG. 3D).

These data demonstrate that proliferation of prostate cancer cells can be decreased by treatment with ERβ ligands, e.g., an antiestrogen. The data also show that cell growth assays can be used to identify compounds.

### Example 7

### Reduction of ICI-Induced Cell Growth Inhibition by ERβ Antisense ODN

DU145 cells when treated with ICI at 1 μM induced a 40% reduction in cell number (FIG. 4, first column). Co-treatment of DU145 cells with ICI and an ERR antisense ODN led to restoration of cell number (p<0.001, FIG. 4, second column) while co-treatments with an ERβ sense ODN or a mismatch ODN (Table 2) did not reverse the ICI-induced effects (FIG. 4, third and fourth columns). These data indicated that the ICI-induced cell growth inhibition (cell death/apoptosis/cell cycle arrest) in DU145 cells is mediated through an ERβ signaling mechanism.

This is a method of testing whether the effects of a candidate compound are mediated by ERβ.

TABLE 1
Primer Sequences for RT-PCR Analysis

| Target gene | Primer sequence | Location | Expected Size |
|---|---|---|---|
| ERβ | ERβ1:5′TGA AAA GGA AGG TTA GTG GGA | nt.230-253 | 528 bp |
| | ACC3′(SEQ ID NO: 1) | | |
| | ERβ2:5′TGG TCA GGG ACA TCA TCA | nt.737-757 | |
| | TGG3′(SEQ ID NO: 2) | | |
| ERα | ERα1:5′TAC TGC ATC AGA TCC AAG GG3′ | nt.41-60 | 650 bp |
| | (SEQ ID NO: 3) | | |
| | ERα2:5′ATC AAT GGT GCA CTG GTT GG3′ | nt.671-690 | |
| | (SEQ ID NO: 4) | | |
| PR | PR-1:5′GAT TCA GAA GCC AGC CAG AG3′ | nt.1817-1836 | 533 bp |
| | (SEQ ID NO: 5) | | |
| | PR-2:5′TGC CTC TCG CCT AGT TGA TT3′ | nt.2330-2349 | |
| | (SEQ ID NO: 6) | | |
| pS2 | PS2-1:5′GGA GAA CAA GGT GAT CTG CG3′ | nt.52-71 | 236 bp 236 bp |
| | (SEQ ID NO: 7) | | |
| | PS2-2:5′CAC ACT CCT CTT CTG GAG GG3′ | nt.268-287 | |
| | (SEQ ID NO: 8) | | |
| AR | AR-1:5′CTC TCT CAA GAG TTT GGA TGG | nt.2896-2918 | 342 bp |
| | CT3′(SEQ ID NO: 9) | | |
| | AR-2:5′CAC TTG CAC AGA GAT GAT CTC | nt.3214-3237 | |
| | TGC3′(SEQ ID NO: 10) | | |
| GAPDH | GAPDH-F:5′CCA CCC ATG GCA AAT TCC ATG | nt.152-175 | 598 bp |
| | GCA3′(SEQ ID NO: 11) | | |
| | GAPDH-R:5′TCT AGA CGG CAG GTC AGG TCC | nt.726-749 | |
| | ACC3′(SEQ ID NO: 12) | | |

TABLE 2
Oligonucleotide Sequences for
Antisense ODN Experiments

| Target | | | |
|---|---|---|---|
| gene | Sequences | Location@ | Size |
| ERβ | 5′C*A*T* CAC AGC AGG GCT | −15 to +3 | 18 bp |
| Antisense | A*T*A*3′(SEQ ID NO: 13) | | |
| Sense | 5′T*A*T* AGC CCT GCT GTG | | |
| Mismatch | A*T*G*3′(SEQ ID NO: 14) | | |
| | 5′G*A*T* CTC AGC ACG GCA | | |
| | A*A*T*3′(SEQ ID NO: 15) | | |

*This base was phosphorothioate-modified.
@The first base of translation-initiating site is +1.

### Example 8

### ERβ Modulation of Gene Expression

cDNA array technology was used to identify genes whose expression is modulated by ICI 182,780 in DU145 cells. The DU-145 cells were treated for 12 hours with ICI 182,780, and total RNA was then isolated from treated cells and untreated controls. Following poly-(A)-RNA selection of these two population of total RNA, and DNase I digestion to remove contaminating DNA, the poly (A)-RNA pools were reversed-transcribed in the presence of P32 to produce radiolabeled cDNA probes. These radiolabeled cDNA probes were applied to an Atlas™ cDNA Expression Array (600 gene array, Clontech Laboratories, Inc, Palo Alto, Calif.) using the vendor's recommended protocol. Seven genes were identified as being up-regulated in response to cell treatment with ICI 182,780. The seven up-regulated genes are listed in Table 3 (below).

TABLE 3
ERβ Regulated Genes

| | Fold | |
|---|---|---|
| | difference | |
| Name of Gene | relative to | |
| (Position on Array) | control | Potential functions |
| RYK receptor-like tyrosine | +3.6 | The human ryk tyrosine found to represent a |
| kinase (E2c) | | ubiquitously expressed gene. Message localization found |
| | | in epithelial and stromal compartment of tissues such as |
| | | brain, lung, colon, kidney, and breast. Increased |
| | | expression in ovarian tumors, and pediatric brain tumors, |
| | | an anaplastic ependymoma, a glioblastoma multiforme and |
| | | a primitive neuroectodermal tumor |
| 5-hydroxytryptamine A1 | +6.6 | It belongs to the G protein coupled serotonin (5- |
| receptor (E4h) | | hydroxytryptamine) receptor subtype. It plays a role in |
| | | mediating the neurotrophic effects of serotonin. Expression |
| | | is detected in midbrain dorsal raphe nucleus and activation |
| | | of the receptor inhibits the firing of serotonin neurons and |
| | | the release of neurotransmitter. In astrocytes it play a rolde |
| | | in the synaptic plasticity necessary for certain experience- |
| | | driven brain changes, such as memory or learning. It |
| | | modulates fear-related behavior. |
| BCL-2 related A1 protein | +9.6 | BFl-1 is an anti-apoptotic member of the BCL-2 family |
| (BFL-1)(C4H) | | The bfl-1 protein suppreses apoptois induced by the p53 |
| | | tumor suppressor protein in a manner similar to other Bc1- |
| | | 2 family such as Bc1-2, Bc1-xL and EBV-BURF1. |
| | | Activation of monocytes leads to dramatic induction of |
| | | Bfl-1. It is abundantly expressed in bone marrow. in |
| | | tumor tissues, its expression was preferentially detected in |
| | | infiltrating inflammatory cells rather than in cancer cells. |
| Embryonic | +4.5 | GDF-1 is a member of the transforming growth factor beta |
| growth/differentiation | | superfamily that is expressed in the brain. In mice GDF-1 |
| factor (GDF-1) | | is most likely an extracellular factor mediating cell |
| | | differentiation events during embryonic development. |
| Interleukin-12 alpha chain | +10.6 | IL-12 promotes cell-mediated immunity by facilitating |
| (natural killer cell | | type 1 helper T-lymphocyte responses, inducing the |
| stimulatory factor, p35) | | secretion of interferon-gamma from both T and natural |
| (IL-12)(F6K) | | killer cells, enhancing the lytic activity of natural killer |
| | | cells, and augmenting specific cytolytic T-lympbocyte |
| | | responses. IL-12 plays an important role in normal host |
| | | defense against infection by a variety of intracellular |
| | | pathogens. Inhibition of IL-12 synthesis may be beneficial |
| | | in diseases associated with pathologic Th1 responses, such |
| | | as multiple sclerosis or Crohn's disease.. It may also have |
| | | clinical utility in the treatment of patients suffering from |
| | | chronic hepatitis B or C virus infections. |
| TL1309 | +2.7 | |
| IFN-α/β receptor | +2.7 | |

Genes regulated by ERβ can be useful e.g., as secondary targets for treating prostate cancer. For example, up-regulation of ERβ-induced genes could be a useful method of regulating prostate cell proliferation.

There are additional reports in the literature of cell lines expressing only ER-β that include genes regulated by this receptor. Examples include the thymidylate synthase gene, survivin gene, telomerase gene, hTERT gene, a subunit of the enzyme telomerase, tumor necrosis factor-alpha (TNF-alpha) and quinone reductase. See, e.g., Nakayama et al., “Tamoxifen and gonadal steroids inhibit colon cancer growth in association with inhibition of thymidylate synthase, survivin and telomerase expression through estrogen receptor beta mediated system,” Cancer Lett., 2000, 161(1):63-71 (PMID: 11078914); Routledge et al., “Differential effects of xenoestrogens on coactivator recruitment by estrogen receptor (ER) alpha and Erβ,” J Biol Chem., 2000, 275(46):35986-93 (PMID: 10964929); Misiti et al., “Induction of hTERT expression and telomerase activity by estrogens in human ovary epithelium cells,” Mol. Cell. Biol., 2000 20(11):3764-71 (PMID: 10805720); An et al., “Estradiol repression of tumor necrosis factor-alpha transcription requires estrogen receptor activation function-2 and is enhanced by coactivators,” Proc. Natl. Acad. Sci., USA 1999, 96(26):15161-6 (PMID: 10611355); Srivastava et al., “Estrogen decreases TNF gene expression by blocking JNK activity and the resulting production of c-Jun and JunD,” J. Clin. Invest., 1999, 104(4):503-13 (PMID: 10449442).

### Example 9

### Identification of Additional ERβ Modulated Genes

The above nucleotide array experiments are repeated using ICI 182,780 and higher density arrays to identify additional ERβ-molulated genes. Such arrays are obtained commercially from sources including New England Nuclear (Boston, Mass.)(>2000 gene array); Research Genetics (Huntsville, Ala.)(>5000 gene array); and Genome Systems Inc. (See Incyte Genomics, Palo Alto, Calif.)(>7,000 gene array). Some of the additional ERβ-molulated genes are up-regulated, e.g., about 2-fold to about 50-fold. In contrast, some of the additional ERβ-molulated genes are down-regulated, e.g., about 2-fold to about 50-fold. The relationship of these genes to the ERβ and apoptosis/cell death/cell cycle arrest pathway is confirmed by analysis of RNA pools isolated from: (1) cells treated with ICI 182,780 and not treated with ERβ antisense oligonucleotide; and (2) cells treated with ICI 182,780 and ERβ antisense oligonucleotide. Genes regulated (or induced) by ICI 182,780 acting via ERβ show reduced response to ICI 182,780 in the presence of the ERβ antisense oligomer.

The following examples describe the generation of an ERβ-specific antibody, and illustrate that prostatic carcinogenesis was characterized by a loss of ERβ expression at the protein and transcript levels in high-grade dysplasias, its reappearance in grade 3 cancers, and its diminution/absence in grade 4/5 neoplasms.

### Example 10

### Generation of an ERβ-Specific Polyclonal Antibody (GC-17)

To make an ERβ-specific antibody, an immunizing peptide was selected with aid of the computer programs Protean (DNASTAR, Inc., Madison, Wis.) and Peptool (BioTools, Inc., Edmonton, AB, Canada). A peptide sequence in the F domain of the human ERβ receptor (amino acids 449-465) was selected, as there is no homology with estrogen receptor alpha (ERα) at this region (Mosselman et al., FEBS, Lett 392:49-53, 1996; Gustafsson J A, Semin Perinatol, 24:66-69, 2000). The peptide was custom synthesized by Research Genetics (Huntsville, Ala.) with a format of 4-branch Multiple Antigenic Peptide. Each rabbit (male NZW, 5-6 lbs) was first inoculated with 0.5 mg peptide antigen with complete Freund's adjuvant, and then boosted with 0.25 mg peptide plus Incomplete Freund's adjuvant at day 14, day 21, and every two weeks afterward until a satisfactory serum titer was obtained. A direct Enzyme-Linked ImmunoSorbent Assay (ELISA) was used to assess the immune responses to the peptide antigen (Harlow et al., A Laboratory Manual, 139-242, 1988).

### Example 11

### Methods Used to Test the Specificity of the GC-17 Antibody

Competitive Inhibition ELISA Assay

The wells of an ELISA plate, PRO-BIND™ (Becton-Dickenson Labware, Franklin, N.J.) were coated with a recombinant protein composed of the entire ERβ sequence (PanVera, Madison, Wis.) at a concentration of 5 μg/ml. The GC17 antibody (1:6000) was then pre-incubated with the immunizing peptide at concentrations ranging from 4 mg-4 μg/ml at room temperature for 30 minutes. In addition, 4 mg of a control peptide encompassing sequences in the N-terminal region of ER-β (Research Genetics) was preincubated with the GC 17 antibody (1:6000) at room temperature for 1 hour. The resulting antigen/antibody complexes were then incubated with the bound recombinant ER-β protein on the ELISA plate at 37° C. for 2 hours. Alkaline phosphatase conjugated anti-rabbit IgG antibody (Jackson ImmumoResearch, West Grove, Pa.) was used to recognize the GC17 antibody, which bound to recombinant ERβ protein on the plate. The whole complexes were visualized by incubation with p-Nitrophenyl phosphate in 2-amino, 2-methyl, 1,3-propanediol buffer pH 9.6. Results were quantified by optical density using the Microplate reader 550 (Bio-Rad, Richmond, Calif.). The entire assay was done four times.

Competitive Immunohistochemistry

Lau et al. (Cancer Res 60:3175-3182, 2000) have recently demonstrated that DU145 and LNCaP cells, both derived from a metastatic prostate cancer, express abundant ERβ mRNA but not ERα message. These cells were used to compliment and confirm that the GC-17 antibody reagent specifically detected ERβ but not ERα by immunohistochemical staining. Using the GC-17 antibody and the anti-ERα antibody (NCL-ER-6F11, Novacastra, Newcastle, UK) at the same dilutions as for tissue sections (see below), we carried out immunohistochemical studies on 10% formalin-fixed cytospins of DU145 and LNCaP cells that had been routinely processed, embedded in paraffin, sectioned at 5 μm and mounted on SuperFrost Plus™ slides (VWF Scientific, West Chester, Pa.).

Peptide competition studies were performed at the immunohistochemical level that approximated the conditions used in the ELISA assays described above. GC-17 antibody, at a dilution of 1:6000, was incubated with the immunizing ERβ peptide at concentrations of 400 μg and 40 μg at room temperature for 1 hour. In addition, competitive studies were conducted using ERα recombinant peptide (400 and 40 μg, Affinity Bioreagents Inc., Golden, Colo.) on DU145 cells. Incubation conditions and time were identical to those used for the ERβ peptide competition studies. Deparaffinized sections of DU145 and LNCaP cells and human prostate tissue were then incubated with these mixtures at room temperature for 1 hour. Competition studies, done on prostate tissues, were identical to those performed on DU145 cells, except the peptide and antibody mixtures were incubated overnight and then applied to sections for 24 hours at room temperature. All of the remaining immunohistochemical and other staining procedures were identical to those used for tissue sections (see immunohistochemical procedures).

Western Blot Analysis

Four human normal prostate tissues from radical prostatectomies and one normal human testis tissue were used in this analysis. In addition, we used normal prostate epithelial cells (Clonetics, walkersville, Md.) and DU145 cells (ATCC, Rockville, Md.) for these studies. Recombinant proteins, ERα (RP310) and short form of ER-β (RP311) (Affinity Bioreagents Inc., Golden, Colo.) as well as long form of ER-β (PanVera, Madison, Wis.), were included as controls. Tissues or cells were homogenized in radioimmunoprecipitation (RIPA) buffer containing 50 mM Tris-HCl pH 7.4, 1% Nonidet P-40 (Amaresco, Solon, Ohio), 0.5% sodium deoxycholate, 0.1% SDS, 1 mM phenoylmethylsulfonyl fluoride (PMSF) in isopropanol, 1 mM activated sodium orthovanadate and 2X Complete™ proteinase inhibitor cocktail (Boehringer Mannheim, Mannheim, Germany). Twenty-five μg of tissue protein extracts, 0.5 μg of recombinant ERα protein or 0.5 μg of recombinant ERβ protein were mixed with 2X SDS loading buffer (125 mM Tris buffer pH 6.8, 20% glycerol, 2% SDS, 2% β-mercaptoethanol and 1 μg/ml bromophenol blue) and electrophoresized onto a 10% SDS-polyacrylamide gel under reducing condition. The separated proteins were transferred onto a PolyScreen® PVDF transfer membrane (NEN, Boston, Mass.). The membrane was incubated for 1 hour in blocking buffer (PBS with 5% nonfat dry milk). The primary antibodies were applied at 1:6000 for GC17 ER-β antibody or 1:5 for 1D5 ERα antibody (Biogenex, Mountainview, Calif.) in PBS-T (PBS with 0.05% Tween-20) buffer with 0.1% bovine serum albumin for overnight at room temperature. After washing 5 times with PBS-T buffer, the membrane was incubated with horseradish peroxidase-linked donkey anti-rabbit IgG antibody (Amersham Pharmacia Biotech, Piscataway, N.J.) for GC17 or goat anti-mouse IgG antibody (NEN, Boston, Mass.) for 1D5 at 1:2500 for 1 hour. The signals were visualized with chemiluminescence ECL detection system (NEN, Boston, Mass.) and autoradiography. All reagents were purchased from Sigma (St. Louis, Mo.) unless specified.

### Example 12

### Preparation of Prostate Tissues

a) Formalin Fixed Radical Prostatectomy Specimens

Tissues studied were from 50 radical prostatectomy specimens collected at Stanford University Medical School, during the years 1995-1999. Patients ranged in age from 46-73 years of age and none had received any treatment prior to their undergoing prostatectomy. Prostates were fixed in 10% buffered formalin for 24 hours then sectioned transversely. Tissues were dissected fixed in 10% buffered formalin for 3 hours, routinely processed, and embedded in paraffin. A histopathological diagnosis was made on a hematoxylin and eosin stained (H&E). The criteria used in the grading of the carcinomas were those described by Stamey et al., JAMA, 281:1395-1400, 1999. The slide, together with the corresponding paraffin block, was then sent to IL where immunohistochemical studies were carried out. At least one section from each case was stained with H&E and reviewed by IL to assure that it matched the tissue components in the original slide. Paraffin sections were cut at 6 μm mounted on SuperFrost™ Plus slides. Sections were left unbaked until used for immunohistochemical studies.

Among the 50 cases selected for study, 26 contained areas of carcinoma. Five of these were clear cell carcinomas of the transition zone while all the remaining cancers were found in the peripheral zone. All of the peripheral zone cancers were composed of mixtures of grade-3 and 4/5 carcinomas. In contrast, all of the clear cell carcinomas were predominately grade-3 neoplasms. Twenty of the peripheral cancer specimens also contained varying amounts of low/moderate to high-grade dysplastic lesions. Dysplasia of the peripheral zone was found in the absence of carcinoma in 6 of the 50 total cases we studied. Additionally, 7 of the 50 cases, were low/moderate grade dysplasias of the central zone that did not coexist with cancer. Among the 50 cases, two specimens each of lesion-free normal peripheral, central and the transition zone were included in our study. Among the cases studied, 15 examples of benign prostatic hyperplasia (BPH) were either commingled with other lesions (Ho et al., CRC Press, 73-113, 1997) or occurred separately (Leav et al., AM J Pathol, 93:69-92, 1978).

b) Bone and Lymph Node Metastases

In addition to the prostatectomy cases, archived paraffin blocks containing bone metastases were obtained from 7 patients, treated at University of Massachusetts Medical Center. The patient's ages ranged from 59-74 and they were all clinically stage D2 at the time of diagnosis. All received antiandrogen treatment as follows: 1) Four patients were orchectomized. One of these patients was given the LH/RH agonist Lupron (TAP Pharmaceuticals inc, Deerfield, Ill.) and the AR competitive inhibitor Eulixin (Flutamide-Schering corp. Kenilworth, N.J.) for 3 months, one treated with Eulixin for 24 months and the remaining two were not given any further antiandrogenic therapy. 2) Three patients were not orchectomized. Two were treated with Lupron for 8 months and the other with Lupron and Eulixin for 3 months. Following these antiandogenic therapies for the periods noted above, it was determined that all seven patients were failing therapy. At those time points, biopsies of suspected bone metastases were obtained from the iliac crest of each patient. These samples were immediately fixed in 10% buffered, routinely processed, embedded in paraffin, 6 μm sections placed on SuperFrost™ Plus slides, and stained with H&E. Replicate sections of these lesions were used for immunohistochemical studies.

In addition, we also studied 5 archived examples of metastases to regional lymph nodes. Two cases were obtained from Department of Pathology at University of Massachusetts Medical School and the remaining 3 were from University of Florida Medical School (a gift from Dr William Murphy). The patients were 60-85 years of age. Regional lymph nodes (external iliac and pelvic) were obtained from all patients during radical prostatectomy. Only one patient had received any treatment prior to surgery (Lupron).

c) Frozen Tissues for LCM/RT-PCR

Eighteen separate specimens, derived from radical prostatectomies, were placed in cassettes containing TBS (Triangle Biomedical Sciences, Durham, N.C.) and quick-frozen in liquid nitrogen. Approximately fifteen minutes elapsed from the surgical removal of the gland to the initiation of freezing. Formalin-fixed and paraffin embedded tissue sections, immediately adjacent to the quick frozen specimens, were also taken for subsequent immunohistochemical studies (see below).

For diagnostic purposes and lesion selection, tissues were first cryostat sectioned and then fixed briefly in 70% ethanol and stained with H&E. The frozen tissue blocks were then stored at −70° C. until they were used for microdissection. Prior to LCM, sections from these cases were found to contain varying amounts of grade three and 4/5 carcinoma as well as dysplastic and normal glands.

### Example 13

### Immunohistochemical Procedures

The followings are the primary antibodies and the dilutions used in our studies:

Anti-estrogen receptor beta (ERβ), rabbit polyclonal antibody GC-17, diluted at 1:6000; anti-estrogen receptor alpha (ERα), mouse monoclonal antibody NCL-ER-6F11, diluted at 1:50 (Novocastra, Newcastle upon Tyne, UK); anti-androgen receptor (AR), rabbit polyclonal antibody, diluted to 22.7 μg/ml (Upstate Biotechnologies, Lake Placid, N.Y.); anti-Mib5/Ki67, mouse monoclonal antibody, diluted at 1:50 (Immunotech, Westbrook, Me.) and anti-high molecular weight cytokeratin (HMWC), mouse monoclonal antibody 34βE12 diluted at 1:50 (Enzo Diagnostics, Farmingdale, N.Y.). Immunostaining for Prostatic Specific Antigen (PSA) was done with a Nexus Immunostainer (Ventana, Tuscon, Ariz.) using prediluted reagents.

Five μm thick sections were cut and mounted on SuperFrost™ Plus slides. Sections were left unbaked until immediately prior to use at which point they were baked for 1 hour at 60° C. After baking, sections were deparaffinized through three changes of xylene and rehydrated through graded alcohols into water. Heat induced epitope retrieval (HIER) was performed by boiling sections in citrate buffer pH 6.0 (pH 6.2 for ERβ) for 15 minutes on a laboratory hotplate. After boiling, sections were removed from the hotplate, allowed to cool at room temperature (RT) for 20 minutes, and were then rinsed thoroughly with water (sections stained for PSA did not require HIER). Sections were then placed in 3% hydrogen peroxide for 15 minutes at RT to block endogenous peroxidase, washed with water, and placed in PBS (Sigma, St. Louis, Mo.). Sections were then incubated with Power Block (Biogenex) nonspecific blocking reagent for 10 minutes at RT to reduce nonspecific staining, washed with water, and placed in PBS. Sections were then incubated with normal goat serum at 1:50 (Vector, Burlingame, Calif.) for 15 minutes at RT. The goat serum was then shaken off and sections were incubated with primary antibodies overnight at 4° C. After overnight incubation, each section received 20 seconds of washing with PBS, 20 seconds of washing with Biogenex Optimax Detergent Wash Solution followed by 10 minutes of washing in PBS on a rotator. Solutions were changed for every eight slides. Following washing, sections were incubated with either Biogenex Mutlilink secondary antibody at a dilution of 1:20 for 20 minutes at RT or DAKO (Carpinteria, Calif.) ready to use secondary antibody for 10 minutes at RT. Sections were again washed according to the protocol described above. Sections were then incubated with either Biogenex Streptavidin-conjugated horseradish peroxidase at a dilution of 1:20 for 20 minutes at RT or DAKO ready to use streptavidin-conjugated alkaline phosphatase for 10 minutes at RT. Sections were again washed as previously described. Immunostaining was visualized using either Biogenex liquid 3,3-diaminobenzidine (Biogenex) or DAKOs New Fuchsin as the chromogen. Following development, sections were rinsed in water, lightly counterstained with 10% Harris Modified Hematoxylin.

Positive controls for GC-17 included DU145 cells and tissue sections of prostate, which were previously shown to be consistently stained with the antibody. Positive tissue controls for ERα were human breast cancers, shown to contain numerous immunostained cells. Morphologically normal human prostate sections served as positive controls for AR as well as for HMWC and MIB5/Ki-67 stains. For all reagents, negative controls were performed by substituting the primary antibody with a class-matched isotype.

### Example 14

### Laser Capture Microdissection (LCM) and RT-PCR

In all instances, immunohistochemical studies for ER-β were performed on the paraffin tissue sections, which are adjacent to the frozen sections used for microdissection and RT-PCR analysis. Frozen sections were cut on a cryostat at 5 μm placed on precleaned glass slides (Fisher Scientific, Pittsburgh, Pa.) and immediately fixed in 70% ethanol for 5 seconds. The sections were then briefly dipped in distilled water, stained with 10% Harris hematoxylin for 15 seconds, dipped in distilled water, then successively placed in 70% ethanol for 30 seconds, briefly immersed in 1% eosin then placed in 95% ethanol 1 minute, two changes of 100% ethanol 1 minutes each, and two changes of xylene 30 seconds each. After air-drying for approximately 30 minutes tissues were microdissected using a Pixcell 2 Laser microdissection unit (Arcturus, Mountainview, Calif.). Eight to ten normal acini were microdissected from each of three different cases. Similarly, 10-20 dysplastic glands were dissected from 4 separate cases of high-grade lesions, and approximately the same numbers of neoplastic glands were obtained from 5 cases of grade 3 and six different examples of grade 4/5 carcinomas.

RNA was extracted from each sample and then separately subjected to RT-PCR analysis. Total cellular RNA was separately isolated using RNA Stat-60 reagent (Tel-Test Inc., Friendwood, Tex.) according to protocols provided by the manufacturer. The total isolated cellular RNA was reverse-transcribed using the GeneAmp RNA PCR kit (Perkin Elmer, Conn.) in total 20 μl reaction mixture and 2 μl of the resulting cDNA was used in PCR on ER-α, AR and GAPDH and 3 μl for PCR on ER-β. Hot start PCR using AmpliTaq Gold DNA polymerase (Perkin-Elmer, Conn.) was employed in all amplification reactions. The enzyme was activated by pre-heating the reaction mixtures at 95° C. for 6 minutes prior to PCR. The PCR programs were 45 cycles for GAPDH and 55 cycles for ER-α, AR and ER-β of 1 minutes at 94° C., 1 minute at 60° C. (annealing temperature) and 1 minute at 72° C. This protocol was chosen to minimize non-specific product amplification.

The primer sequences for ERα, AR and GAPDH were described in our previously study (Lau et al., Cancer Res, 60:3175-3182, 2000). The primer set for ERβ was newly designed and the forward primer is 5′-GATGAGGGGAAATGCGTAGA-3′(SEQ ID NO: 16) and the reverse primer is 5′-CTTGTTACTCGCATGCCTGA-3′(SEQ ID NO:17).

### Example 15

### Specificity of the GC-17 Antibody

Competitive ELISA

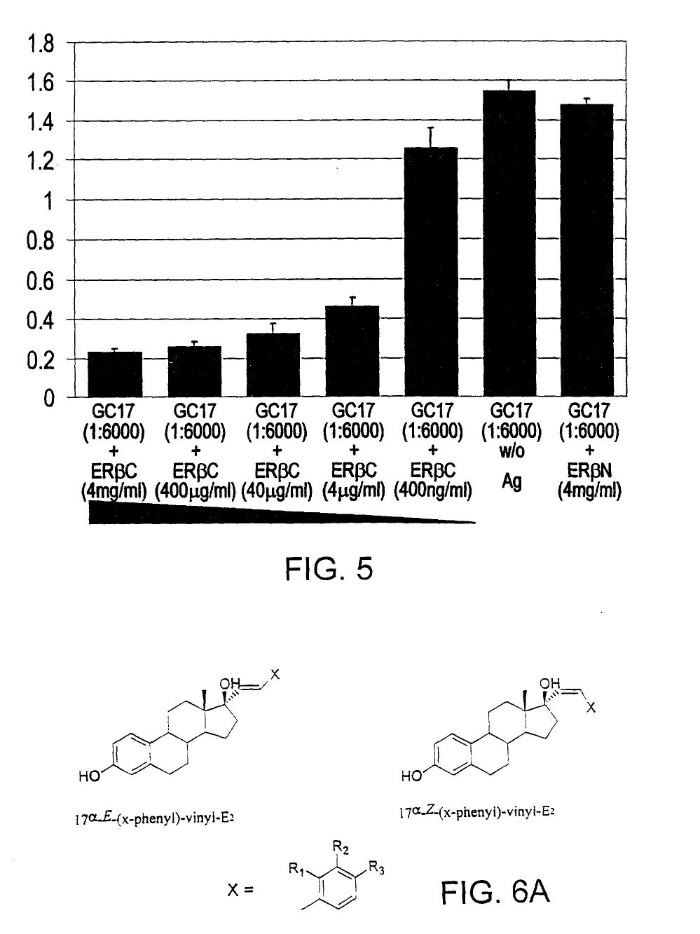

Pre-incubation of GC-17, an antibody raised against the F domain of ERβ, with the immunizing peptide (C-terminus of ERβ, ERβC) successfully suppressed binding to the recombinant protein FIG. 5. The suppression occurred in a concentration-dependent manner when compared to the control where the antibody was not pre-incubated with the immunizing peptide. In contrast, pre-incubation of GC-17 with the control N-terminus peptide of ERβ (ERβN; 4 mg/ml) did not significantly suppress binding when compared with the control, indicating that the antibody was not cross-reactive with this region of the ERβ protein.

Competitive Immunochemistry

Strong nuclear immunostaining was detected in sections of DU145 and LNCaP cells which served as positive controls for the peptide competition studies. Pre-incubation of GC-17 with either 400 μg/ml or 40 μg/ml of the immunizing peptide ERBC totally abolished nuclear staining in sections of these cells, when compared with positive controls where the peptide was omitted. Identical results were obtained with sections of human. Preincubation of GC17 with the recombinant ERα protein failed to block ERβ immunosatining of DU145 cells by the antibody. These studies confirmed that GC-17 does not cross react with ERα and supports data from the Western Blot findings (infra). Thus, both the competitive ELISA and competitive immunohistochemistry showed GC-17 to be highly specific for binding to the ERβ protein.

In addition, DU145 and LNCaP cells, that only express ERβ, Lau et al. (Cancer Res 60:3175-3182, 2000), were negative when immunostained with the ERα (NCL-ER-6F11) antibody. Positive staining of prostate tissues with the ERα antibody was restricted to stromal cells (see immunohistochemistry of normal prostate below).

Western Blot Analysis

Using Western blot analysis, GC-17 was demonstrated to specifically recognize two recombinant ERβ proteins and show no cross-reactivity to ERα protein. The size of recombinant ERβ protein (RP311) from Affinity Bioreagents Inc. (Golden, Colo.) is approximately 531 kDa and it represents a short form of ER-β protein using a reported initiator codon (corresponding to 43-530 Kuiper et al., Proc Natl Acad Sci USA, 93:5925-5930,1996; Mosselman et al., FEBS Letter, 392:49-53, 1996; Tremblay et al., Mol Endocrinol, 11:353-365, 1997 amino acids). The long form of ERβ recombinant protein (PanVera, Madison, Wis.), containing 530 amino acids, was also recognized by GC-17 antibody and showed a 59 kDa band in the blot. The native long form of ERβ protein in the cells is approximately 63 kDa. GC17 recognized an approximately 63 kDa protein in human normal testis and prostate tissues, suggesting that the long form may be the natural ERβ protein in human tissues. The size difference between long form of recombinant ER-β protein and native protein in cells may be related to the occurrence of post-translational modifications in cells and tissues. The level of ERα protein in human normal testis and prostate tissues was undetectable with the 1D5 human antibody after a 30 s econu exposure to X-ray film. However, very weak ERα protein signal was detected only when the blot was exposed to an X-ray film for over 10 minutes.

### Example 16

### Immunohistochemistry of Prostate Tissues

Normal Prostate

In morphologically normal ducts and acini, nuclear ERβ expression was consistently densely localized in nuclei of basal cells as defined by HMWC staining in replicate sections). Strong nuclear staining was absent in secretory cells but frequently observed in stromal cells. Occasionally nuclear membrane staining for the receptor was also evident in a few luminal cells. ERα immunostaining was not present in secretory cells of normal ducts and acini but individual scattered receptor positive basal cells were observed in less than 10% of all sections studied. The ERα receptor was, however, consistently found in stromal cell nuclei, especially in periglandular locations.

Pronounced nuclear staining for AR was a constant finding in secretory and stromal cell nuclei. In agreement with a past study (Gustafsson et al., Semin Perinatol, 24; 66-69, 2000), variable immmunostaining for AR was also observed in individual basal cells of normal glands. No difference was found in the cellular localization of the three steroid hormone receptors when the peripheral, transition, and central zones of the prostate were compared. Moreover, the same localization of the steroid hormone receptors, found in the three normal zones, was also seen in foci of BPH.

The most consistent immunolocalization for the three receptors in basal cells was found within periurethral ducts.

Dysplastic Lesions

Immunohistochemical findings in dysplastic and carcinomatous lesions are summarized in Table 4.

TABLE 4
Immunohistochemical finding in dysplasias and carcinomas

| Lesion | ER-α | ER-β | AR |
|---|---|---|---|
| Dysplasia/Peripheral zone | | | |
| Moderate grade* | − | + | + |
| High grade | − | − | + |
| Dysplasia/Central zone* | + | + | + |
| Carcinomal/Peripheral zone | | | |
| Grade3 | −/+ | + | + |
| Grade 4/5 | + | −/+ | + |
| Carcinoma/Transition zone | − | − | + |
| Metastic carcinoma | | | |
| Bone | − | + | + |
| Lymph nodes | −/++ | + | + |

*Data reflects positive staining in dysplastic and basal cells. Staining restricted to residual basal cells in high-grade dysplasia.
“+” = Positive staining in >50% of cases;
“−/++” = Positive staining in 40% of cases;
“−/++ = Positive staining in <20% of cases. See text for estimates of the % of positively stained cells in each lesion.

In the peripheral zone, a consistent pattern of ERβ expression was found in dysplastic lesions. A secretory cell localization for nuclear ERβ expression was commonly observed in low to moderate grade dysplasitic lesions. The majority of both basal and dysplastic secretory cells in these lesions, contained moderate to strongly stained nuclei. A marked diminution to total absence of ERβ immunostained nuclei was a consistent feature in almost all dysplastic cells in the high grade lesions that were studied. Staining was however present in residual basal cells within these high-grade lesions. Thus, a loss of ER-β staining in high-grade dysplasias paralleled a decline in receptor-positive basal cells. In contrast to normal cells, the cytoplasm of dysplastic cells in lesions of all grades but especially in high grade lesions were frequently stained by the GC-17 reagent, a feature not seen when the primary antibody was omitted from the incubation or with the use of any of the other antireceptor reagents. Similarly nuclear membrane of cells in high grade dysplastic cells also frequently stained with the GC-17 antibody. ER-α positive dysplastic cells were not present within any of the peripheral zone lesions thatwere studied.

In marked contrast, ER-α stained cells were detected in 5 of 7 (71%) of dysplasias in the central zone. There was great variation in the numbers (10-90%) of immunopositive cells in any given central zone lesion. In replicate sections, 6 of these lesions contained dysplastic cells that were also positive for ER-β staining.

As previously reported (Leav et al., Prostate, 29:137-145, 1996), AR was strongly expressed in the -majority (>95%) of dysplastic cells irrespective of the origin or grade of the lesion.

Grade 3 and 4/5 Carcinomas

A transition from ER-β positive to negative staining of cells was observed in all 21 cases where cancer was found in the peripheral zone that paralleled the progression of the grade 3 carcinomas to the less differentiated grade 4/5 neoplasms. ERβ positive cells were found in 13 of 15 (87%) grade 3 carcinomas of the peripheral zone. The spectrum of expression ranged from examples where all nuclei in an individual neoplastic gland were strongly stained to instances where receptor immunostaining was weak and/or found in few cells within a given microscopic field. The latter examples were most often located in areas where a transition to higher-grade carcinoma occurred. Unlike their counterparts in the peripheral zone, the vast majority of cells comprising grade 3 clear cell carcinomas in the transition zone were devoid of ERβ immunostaining. In two of five cases, scattered receptor positive neoplastic cells were found in a minority of glands.

An almost complete absence of ERβ nuclear staining was seen in all but 3 of 15 (20%) grade 4/5 carcinomas. In the majority of cases however staining of the nuclear membrane was frequently apparent. In the 3 cases, positive cells represented only 10% or less of the total cancer cells in a given lesion and the staining intensity was usually diminished.

Immunostaining for ERα was detected in only two (7.6%) of the total twenty-six cases of primary carcinoma we studied. In these two instances, a few (<10%) weakly positive cells were found in both grades 3 and 4/5 carcinomas. Staining for the receptor was consistently absent in all clear cell carcinomas of the transition zone.

Irrespective of grade, strong nuclear AR immunostaining was a constant feature in the vast majority cells (>95%) comprising cancers of the peripheral zone. Nuclear AR immunostaining was also present in almost all grade 3 clear cell carcinomas but it was less intense than found in peripheral zone carcinomas. Interestingly, the occasional few cells that were ER-β positive in these cancers were found to be negative for AR expression in replicate sections.

No change in the location or intensity of immunostaining for the three receptors in the stroma was evident in sections that contained carcinoma.

Metastatic Lesions

Nuclear ERβ immunostaining was present in metastatic carcinoma cells in bone lesions from all but one of the seven cases. The intensity of signal did vary from strong to weak staining within cells comprising the lesions of individual case and/or among the cases. In three instances, carcinoma cells were surrounded by a prominent desmoplastic response in which ERβ staining was frequently found in the nuclei of fibroblasts. Nucleated hematopoietic marrow cells were also positive for the receptor while mature red cells were negative, a finding that served as a positive and negative internal tissue control for ERβ immunostaining in these lesions.

In contrast to the finding of ERβ immunostaining in foci of all but one case of bone metastasis, no staining was observed for ERα in these lesions. Despite the fact that all of seven patients, including the one that lacked ERβ expression had been given antiandrogenic therapy, nuclear AR staining was observed in most metastatic cells in the bone biopsies. However, AR staining was also consistently present in the cytoplasm as well as in the nucleus of metastatic cells, a finding observed in bone and lymph node lesions from all patients who received antiandrogenic therapy (see below).

PSA immunostaining was found in metastatic bone lesions in four cases but it tended to be scant especially when compared to lesions in lymph nodes from patients who did not receive antiandrogenic therapy (see following discussion).

Among the five cases of lymph node metastases, two contained a majority of neoplastic cells (>50%) that were uniformly strongly stained for ERβ. In one case, immunostaining for the receptor was absent in the metastatic cells while in the remaining two cases a mix of negative and positively stained neoplastic cells were found. In one of the two cases, where strong receptor expression was detected, the patient had been treated with the LH/RH agonist Lupron prior to surgery. Lymphocyte nuclei were consistently stained for the receptor and served as an internal positive tissue control in the two cases where no receptor was detected in metastatic cells within the lymph nodes.

ERα immunostained carcinoma cells were found in 2 of 5 cases of lymph node metastases. Both patients were untreated prior to surgery. In one of these cases, ERβ staining was absent in metastatic cells. In one case, numerous ERα positive cells (>50%) were mixed with those in which receptor expression was absent. Very weak staining for ERα was identified in a few cells (<10%) in the other case.

In all five cases, AR immunostaining was present in the vast majority (>95%) of metastatic cells. Nuclear AR expression was always strong in cancer cells and with the exception of the case where the patient received Lupron, cytoplasmic staining was not present in any of the metastatic cells. Strong PSA immunostaining was present in the cytoplasm of most metastatic cells in all cases including the one where the patient had received antiandrogenic therapy.

### Example 17

### LCM/RT-PCR Analysis

Findings with RT-PCR analysis for ERβ mRNA on LCM lesions approximated the results of immunohistochemical studies done on paraffin sections immediately adjacent to the frozen specimens used for RT-PCR as well as on other cases. ERβ transcripts were detected in 2 of the 3 samples of normal prostatic acini. In contrast, receptor message was found in only 1 of 4 microdissected samples of high-grade dysplasias. Sixty percent of grade 3 carcinomas contained ERβ transcripts while receptor message was detected in 2/6 (30%) of the grade 4/5 cancers. In two cases where ERβ message was present in grade 3 lesions, grade 4/5 carcinomas sampled within the same section, lacked receptor mRNA expression. In close agreement with our immunohistochemical findings, AR mRNA was present in all normal glands, dysplasias, grade 3 cancers, and all but one of the grade 4/5 carcinomas. ERα transcripts were not detected in any of the microdissected specimens that were studied.

The data demonstrate that a consistent pattern of lost ERβ expression at both the transcriptional and translational levels occurs during prostatic carcinogenesis and tumor progression. Thus, there may be loss of an important role that the receptor would normally play in inhibiting growth of the prostate which contributes to neoplastic development. The continued expression of AR throughout these processes, as well as other undefined factors, may therefore exert persistent unopposed growth stimulus acting on these cells.

### Example 18

### Cytotoxicity Studies with Phenyl Vinyl Substituted Estrogens in DU-145 Cells

Candidate compounds can be tested for their ability to modulate cell proliferation in cells that express functional ERβ. The following illustrates this testing method using DU-145 cells.

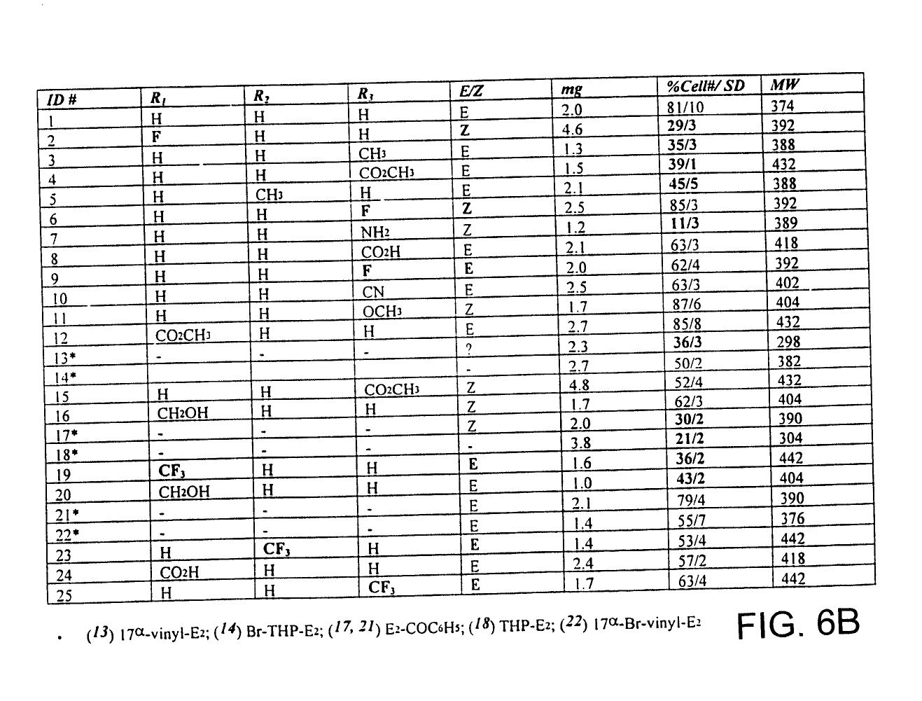

DU-145 cells were treated for 4 days in media containing 1 mM of each of the above reagents after pre-seeding for 2 days in 10% FBS. Various concentrations of Resveratrol® and ICI-182780 were used as positive controls to confirm previous results, i.e., that these compounds lead to a decrease in cell number of 50-70% at 1 mM respectively under the same culture conditions. The percent cell number after treatment was normalized to cells treated with carrier only. FIG. 6A shows the compounds used, and FIG. 6B shows the results of an experiment in which 17α-substituted (e.g., phenyl vinyl substituted) estrogens were used in the assay. These data show that the various compounds listed in FIG. 6B range in their ability to inhibit cell proliferation. FIG. 6A shows the structural formulas of the 17α-E (or Z)-(x-phenyl)-vinyl-E₂, and the structural formula for X. The chart in FIG. 6B shows the results for the 25 different compounds in which the R₁, R₂, and R₃ groups of X are varied, and the E or Z form is designated.

Information can be obtained from such assays regarding not only the ability of the tested candidate compounds to modulate cell proliferation, but also information that is useful for designing additional candidate compounds. For example, examination of the data in FIG. 6B shows that there is a general trend for fluorine labeled compounds to have increased efficacy as inhibitors of cell proliferation in those compounds where the fluorine substitutes are “in close proximity” to the 17β-hydroxy group.

A dose response curve can also be generated using an assay such as the cell proliferation assay described above. Flow cytometry (e.g., analysis of DNA density, allexin V/lactate dehydrogenase) can be used to discern pro-apoptotic from cell static mechanisms of inhibition of cell proliferation. Candidate compounds can also be tested in a quinone reductase assay to assess antioxidant response induced gene expression.

Raloxifene® and Resveratrol® may also be active tumor suppressing agents utilizing the ERβ pathways. Using DU145 and PC-3 M cells that express only ERβ, we found that Raloxifene, an antiestrogen marketed as Evistar by Eli Lily, and Resveratrol, a phenolic compound found in grape skin and seeds, produced marked inhibition of cell growth. The efficacy of Raloxifene is markedly better than that of Resveratrol and ICI. Treatment protocols and dosages used were similar to those described above.

### Example 19

### ERα and ERβ Expression in Normal and Neoplastic Testicular Tissue

Estrogen exposure has been a postulated etiologic factor in the development of testicular neoplasms, and epidemiologic studies have associated prenatal estrogen exposure with an increased risk for germ cell cancer. To date, however, estrogen receptors have not been identified in germ cell neoplasms. The recent identification of the novel estrogen receptor (ER) beta prompted us to investigate if it is present in germ cell malignancies and thus might facilitate the suspected carcinogenic effects of estrogens.

Thirty-two archived surgical specimens from men undergoing radical orchiectomy for testicular cancer were evaluated for the presence of ERβ. Five normal specimens were also examined. All specimens were examined by immunohistochemical stains with both the highly specific anti-human estrogen receptor beta (ERβ) antibody (as described supra) and with commercially available ER alpha (ERα) antibody.

In normal testes, ERβ was localized to the nucleus and highly expressed in spermatogonia and spermatocytes. ERα was not expressed in any testicular specimen, but was noted in adjoining epididymis in two samples. Ninety percent of the seminomas had high expression of ERβ, the remaining specimen (10%) demonstrated low expression. Forty-two percent of MGCTs with embryonal elements had high expression of ERβ, whereas 58% had low expression. All five MGCTs with yolk sac elements and all three cases with teratomatous elements had high expression of ERβ. Both intratubular germ cell neoplasia specimens demonstrated high expression of ERβ. There was an absence of ERα in both normal and malignant testes.

These data provide the first demonstration of the expression of ERβ in testicular germ cell tumors. These findings now suggest a potential mechanism for estrogen's putative involvement in germ cell neoplasia. The differential qualitative expression seen in embryonal cells (MGCTs with embryonal elements) shows that assay of ERβ may be of prognostic significance and be useful for determining the appropriate treatment for certain testicular cancers.

### OTHER EMBODIMENTS

A number of embodiments of the invention have been described. Nevertheless, it will be understood that various modifications may be made without departing from the spirit and scope of the invention. For example, ERβ-regulated genes can be identified using methods other than oligonucleotide arrays, and ERβ-regulated genes other than the seven listed above can be used to screen libraries of compounds to identify candidate compounds that modulate ERβ-mediated cell growth inhibition. Accordingly, other embodiments are within the scope of the following claims.

What is claimed is:

1. An in vitro screening method for identifying a compound that modulates ERβ-mediated cell growth inhibition, the method comprising:
(a) providing a mammalian test cell comprising a functional ERβ protein;
(b) contacting the test cell with a candidate compound;
(c) detecting an increase or decrease in the expression of an ERβ-regulated gene in the presence of the candidate compound, compared to the level of expression of the gene in the absence of the candidate compound, as an indication that the compound modulates ERβ-mediated cell growth inhibition.

2. The method of claim 1, wherein the compound that modulates ERβ-mediated cell growth inhibition promotes cell growth inhibition.

3. The method of claim 1, wherein the compound that modulates ERβ-mediated cell growth inhibition inhibits cell growth inhibition.

4. The method of claim 1, wherein the test cell comprises no detectable ERα protein.

5. The method of claim 1, wherein the test cell is derived from a cell selected from the group consisting of a prostate cell, a neuronal cell, an ovarian cell, a breast cell, a cardiovascular cell and a bone cell progenitor.

6. The method of claim 5, wherein the test cell is derived from a prostate cell.

7. The method of claim 1, wherein the test cell contains an exogenous ERβ gene.

8. The method of claim 1, wherein the test cell contains an exogenous estrogen receptor co-regulator.

9. The method of claim 1, wherein the ERβ-regulated gene is selected from the group consisting of receptor-like tyrosine kinase (RYK), 5-hydroxytryptamine A1 receptor (E2c), BCL-2 related A1, embryonic growth/differentiation factor, IL-12, TL1309, and IFN-αβ receptor.

10. The method of claim 1, further comprising a reporter gene coding sequence operably linked to an ERβ-regulated gene expression control element.

11. The method of claim 1, wherein detecting an increase or decrease in the expression of an ERβ-regulated gene is performed using an oligonucleotide array or a subtractive hybridization technique.

12. A method of identifying a compound that modulates ERβ-mediated cell growth inhibition, the method comprising comparing the proliferation in a cell comprising a functional ERβ protein in the presence and absence of a candidate compound, wherein a decrease in cell proliferation indicates that the compound inhibits cell growth inhibition.

13. The method of claim 12, wherein the cell does not express ERα.

14. A method of identifying a candidate compound that modulates ERβ-mediated cell growth inhibition, the method comprising
(a) comparing cell proliferation in a first cell comprising a functional ERβ protein in the presence and absence of a candidate compound;
(b) selecting the compound if there is a decrease in cell proliferation in the presence of the candidate compound; and
(c) comparing cell proliferation in a second cell that does not express functional ERβ protein in the presence and absence of the candidate compound, wherein no difference in cell proliferation in the second cell indicates that the compound modulates ER-mediated cell growth inhibition.

15. The method of claim 14, wherein the second cell is the same cell type as the first cell.

16. The method of claim 14, wherein the second cell comprises an antisense ERβ oligonucleotide.

17. A compound identified by the method any of claim 1.

18. A method of determining whether a cell is susceptible to ERβ-mediated cell growth inhibition, the method comprising
(a) providing a cell, and
(b) testing the cell for the presence of ERβ, wherein the presence of ERβ indicates that the cell is susceptible to ERβ-mediated cell growth inhibition.

19. The method of claim 18, wherein the cell is a cancer cell.
